(19) United States
(12) Patent Application Publication
(10) Pub. No.: US 2020/0017518 A1
(43) Pub. Date: Jan. 16, 2020

(54) Bis-Cyclic Carbonate Molecule with Bis(1,3)-Dioxin-Dione Structure

(71) Applicant: ChondroNest SA, Conthey (CH)

(72) Inventor: Thierry Schwitzguébel, Conthey (CH)

(21) Appl. No.: 16/469,012

(22) PCT Filed: Dec. 14, 2017
(86) PCT No.: PCT/IB2017/057950

§ 371 Date: Jun. 12, 2019

(30) Foreign Application Priority Data
Dec. 16, 2016 (IB) PCT/IB2016/057707

(51) Int. Cl. C07D 493/10

(52) U.S. Cl. CPC C07D 493/10

(57) ABSTRACT

Bis-cyclic carbonate molecule consisting of at least two (1,3)-dioxine-2-one structures directly linked to each other or indirectly linked via other cycles.

### FIELD OF INVENTION

The invention relates to molecules that may be advantageously used in different applications, such as in the production of novel polymers like polyhydroxyurethane, polycarbonate, the modification of other molecules or the manufacture of solid support media for battery conducting elements and electrical devices.

### STATE OF THE ART

Cyclic carbonates are useful molecules for the production of polymers, co-polymers, or the modifications of other molecules or as solvents, such as in Lithium batteries. Bis cyclic carbonates with five member rings are known and widely used for the production of isocyanates free polyurethanes, polyhydroxyurethanes (PHU), polycarbonates and mixed polymers with esters, lactones, acids, alcohols or thiols. The relative stability of the five members ring carbonate structure requires activation energy or catalysis to obtain the desired products.

Bis Five members rings cyclic carbonate molecules or Six member rings cyclic carbonate molecules carrying a second functionality or linked to each other by a linear side chain are known and have been reported (“Bis(cyclic carbonate) based on d-mannitol, d-sorbitol and di(trimethylolpropane)”, Magdalena, M. Mazurek-Buzynska, ref jeuropolymj.2016.04.021; Pharmaceutical compositions containing functionalized triblock copolymers, INGELL TECHNOLOGIES HOLDING B.V., EP Application Number 14853181; METHOD FOR PRODUCING CYCLIC CARBONATES, Hatti-Kaul Rajni, EP Application Number 14117470; Mono- and Bifunctional Six-membered Cyclic Carbonates Synthesized by Diphenyl Carbonate toward Networked Polycarbonate Films, Hiroyuki Matsukizono, Takeshi Endo) for numerous applications such as for the synthesis of non-isocyanate polycarbonates or polyurethane.

Nothing in the current state of the art describes and characterizes the existence of Spiro-Bis(1,3)-Dioxine-2-one structure such as for example 2,4,8,10-tetraoxaspiro[5,5]undecane-3,9-dione or Bis[(1,3)-Dioxine-2-one] structure fused to sugars or anhydroglucitols such as for example but not limited to Tetrahydro-4H,5H-furo[2,3-d:5,4-d′]bis[1,3]dioxine-2,7-dione and their use for the production of novel and useful polymers such as polyhydroxyurethane, polycarbonate, or polymers with amines, acids, alcohols, lactones and thiols; the modification of proteins and peptides and glycosaminoglycans and proteoglycans or the manufacture of solid support media for battery conducting elements and electrical devices to name some.

### GENERAL DESCRIPTION OF THE INVENTION

The problems mentioned in the production of Polycarbonates, Polyhydroxyurethanes, or polymers with amines, acids, alcohols, lactones and thiols; modified proteins and peptides, modified Glycosaminoglycans and proteoglycans or the production of solid support media for battery conducting elements and electrical devices to name some can be solved with the use of Bis[(1,3)-Dioxine-2-one] molecules described in the present invention.

The invention generally concerns new molecules carrying two or more 1,3-Dioxine-2-one internal structures linked to them or other ring structures such as anhydro glucitols, 2,2,5,5-Tetrakis-Hydroxymethyl Cyclopentanone, 4-Hydroxytetrahydropyran-3,3,5,5-Tetramethanol, 2,2,6,6-Tetramethylol cyclohexanol, Galactopyranose, Lactulose and sugars. Those six members rings cyclic carbonate structures in the same molecule with no alkyl intermediary chain give them useful and unique characteristics. They find for example application in the production of novel polymers such as polyhydroxyurethane, polycarbonate, combination of both, the modification of proteins and peptides and glycosaminoglycans and proteoglycans or the manufacture of solid support media for battery conducting elements and electrical devices to name some.

Generally, the six member rings carbonates reacts at lower temperatures than their five member rings counterparts. They are therefore more useful for reactions at ambient and moderate temperatures. The Bis six member rings cyclic molecules described in this invention allow the preparation of new polymers and co-polymers, the functionalizing of other molecules such as proteins, peptides, glycosaminoglycans and proteoglycans in mild conditions and the preparation of novel solid media for the production of batteries and electrical devices.

The Bis[(1,3)-Dioxine-2-one] react with themselves to produce branched polycarbonates and branched hydroxy polycarbonates by either anionic, cationic or complexation type means.

Reaction with amines, acids, alcohols, lactones and thiols gives unique access to new polymers.

Those new molecules are also useful for the modification of proteins and peptides for example but not limited to their glycosilation or functionalizing. They also open new possibilities to modify Glycosaminoglycans and proteoglycans.

With mono-, di- or poly-primary and secondary amines they form hydroxycarbamates and new polyhydroxyurethanes.

With ions, for example but not limited to Zinc or Lithium, they show an improved behavior compared to mono carbonates such as diethylcarbonate or propylene carbonate due to their unique spatial conformation particularly, but not limited to the molecule 2,4,8,10-tetraoxaspiro[5,5]undecane-3,9-dione.

### DETAILED DESCRIPTION OF THE INVENTION

Carbonates and cyclic carbonates are very interesting functions in organic chemistry. They find applications in a large variety of fields such as polymers, solvents and organic synthesis.

In this family of carbonates, cyclic carbonates with six members ring are of particular interest as they have a specific conformation and internal energy which allow them to exhibit unique reactive properties and unique solvatation properties.

For long people have tried to produce molecules carrying two six members rings on the same molecule. This has been partially resolved through complex synthesis of one six members ring cyclic carbonate with side chain and their attachment to a second six members ring cyclic carbonate with a side chain.

The problem to be solved to achieve new useful properties in polymer science, solvent behavior and organic synthesis is to have molecules with two six members ring cyclic carbonate directly linked to each other or fused to each other via organic cyclic entities.

The present invention solves this problem with new molecules having such Bis[(1,3)-Dioxine-2-one] structures such as for example but not limited to 2,4,8,10-tetraoxaspiro[5,5]undecane-3,9-dione or Tetrahydro-4H,5H-furo[2,3-d:5,4-d′]bis[1,3]dioxine-2,7-dione. Similar fused Bis[(1,3)-Dioxine-2-one] can be produced from anhydro glucitols, 2,2,5,5-Tetrakis-Hydroxymethyl Cyclopentanone, 4-Hydroxytetrahydropyran-3,3,5,5-Tetramethanol, 2,2,6,6-Tetramethylol cyclohexanol, mono saccharides for example but not limited to Galactopyranose, or disaccharides for example but not limited to Lactulose or saccharose.

Those molecules can be produced by classic and known chemical synthesis path such as the use of chloroformates, more particularly Trichloroethylchloroformate or alternatively with phosgene, di and triphosgene or Carbonyldiimidazole.

Those Bis[(1,3)-Dioxine-2-one] molecules allow the production of polymers for example but not limited to polymers with amines, acids, alcohols, lactones and thiols having increased hydrophilicity and good mechanical resistance.

Those Bis[(1,3)-Dioxine-2-one] molecules allow the modification of proteins and peptides such as for example but not limited to glycosilation, side chain attachment or bioavailability modifications or physical properties modification.

Those Bis[(1,3)-Dioxine-2-one] molecules allow the modification of proteoglycans and glycosaminoglycans such as for example but not limited to glycosilation, side chain attachment or bioavailability modifications or physical properties modification.

Those Bis[(1,3)-Dioxine-2-one] molecules allow the creation of novel solid support for example, but not limited to novel solid media for the production of batteries and electrical devices. More particularly, due to its unique spatial conformation, the 2,4,8,10-tetraoxaspiro[5,5]undecane-3,9-dione can, for example but not limited to, be co-crystalized with ions such as but not limited to ionic form of Zinc, Lithium, Sodium, Iron, Lead, Cadmium to name some.

### EXAMPLE 1

100 mM of finely grounded Pentaerythritol is added to 350 ml anhydrous diethylene glycol diethylether. A solution of 202 mM of 2,2,2-Trichloethyl Chloroformate in anhydrous diethyleneglycol dimethylether is added drop wise under agitation over a period of around 8 hours with a constant flow of Argon through the reactor. The mixture is heated between 160 and 170° C. and stirred for 10 to 60 hours until the distillate does not contain HCl. Anhydrous diethylene glycol diethylether is added to keep the reaction volume around 300-350 ml.

The reaction mixture is cooled at around zero degree and the product 2,4,8,10-tetraoxaspiro[5,5]undecane-3,9-dione precipitates. It is filtered and washed diethylether.

Around 75 mM of solid 2,4,8,10-tetraoxaspiro[5,5]undecane-3,9-dione was obtained. The product has a single 1H-NMR signal at 4.4 ppm in DMSO and C13-NMR signals at 147(s) (Carbonyl), 68.8(s) and 39.5(m) (Quaternary).

### EXAMPLE 2

20 mM of 2,5-anhydro glucitol prepared accordingly to for example the process described in U.S. Pat. No. 3,480,651 dated 25 Nov. 1969 is added to 200 ml anhydrous Dioxane. A solution of 40 mM of 2,2,2-Trichloethyl Chloroformate in anhydrous Dioxane is added drop wise under agitation in an Argon blanketed reactor. The mixture is heated at around 80° C. and stirred for 10 to 60 hours.

The solution is cooled to 15-20° C. and a solution of 41 mM of Triethylamine in anhydrous Dioxane is added drop wise over a period of 4 hours. The reaction mixture is heated up between 80 and 100° C. for 3 hours with a slight current of Argon to remove the excess of Triethylamine.

The reaction mixture is cooled at room temperature and filtered to remove the Triethylamine chlorhydrate and recover 200 ml of clear Dioxane solution of 20 mM of modified 2,5-anhydro glucitol.

The reaction mixture is concentrated under vacuum and cristalised in 2-Methyl-Tetrahydrofuran, the product Tetrahydro-4H,5H-furo[2,3-d:5,4-d′]bis[1,3]dioxine-2,7-dione is recovered.

### EXAMPLE 3

5 mM of the 2,4,8,10-tetraoxaspiro[5,5]undecane-3,9-dione obtained in the example 1 is dissolved in THF. The solution is poured drop wise at room temperature in a THF solution containing 5 mM of the diamine, 1,4-Bis(3-aminopropyl)piperazine. The Polyhydroxyurethane formed in seconds is a water swelling polyhydroxyurethane.

### EXAMPLE 4

10 mM of the 2,4,8,10-tetraoxaspiro[5,5]undecane-3,9-dione obtained in the example 1 is dissolved in 1,4 Dioxane. We add to the solution 20 mM of Zinc Iodide. The mixture is poured on a glass plate and dried.

The dried thin solid film formed with a strong attachment to the glass surface exhibits an electric potential and an electrical surface current.

1: Bis-cyclic carbonate molecule consisting of at least two (1,3)-dioxine-2-one structures directly linked to each other or indirectly linked via other cycles.

2: Molecule of claim 1 condensed in a spiro configuration.

3: Molecule of claim 2 which is 2,4,8,10-tetraoxaspiro[5,5]undecane-3,9-dione.

4: Molecule of claim 1 fused on a Tetrahydrofurane ring from anhydro glucitol.

5: Molecule of claim 4 wherein the two (1,3)-dioxine-2-one structures include Tetrahydro-4H,5H-furo[2,3-d:5,4-d′]bis[1,3]dioxine-2,7-dione.

6: Molecule of claim 1 fused or attached in a spiro configuration to pentane, hexane or pyranose.

7: A method for using the molecule of claim 1 for the production of polyhydroxyurethanes.

8: A method for using the molecule of claim 1 for the production of polycarbonates.

9: A method for using the molecule of claim 1 for the production of copolymers with electrophilic functions.

10: A method for using the molecule of claim 1 for the modification of proteins and peptides.

11: A method for using the molecule of claim 1 use for the modification of Glycosaminoglycans and proteoglycans.

12: A method for using the molecule of claim 1 useful for the production of batteries and electrical devices.

13: The method of claim 9, wherein the electrophilic functions include at least one of amines, acids, alcohols, lactones, and thiols.
